(19) United States
(12) Patent Application Publication
(10) Pub. No.: US 2019/0160138 A1
(43) Pub. Date: May 30, 2019

(54) DIETARY SUPPLEMENT FOR GLUTEN REACTION

(71) Applicant: The Remedy Lab, LLC

(72) Inventors: Shannon CONWAY, Bend, OR (US); Pamela MACFETRIDGE, Seattle, WA (US)

(21) Appl. No.: 16/071,064

(22) PCT Filed: Jan. 18, 2017
(86) PCT No.: PCT/US2017/014001

§ 371 Date: Jul. 18, 2018

Related U.S. Application Data
(60) Provisional application No. 62/280,649, filed on Jan. 19, 2016.

(51) Int. Cl. A61K 36/9066; A61P 25/22; A61P 29/00; A61P 1/00; A61P 37/00; A61K 9/14; A61K 9/48; A61K 36/752; A61K 36/67

(52) U.S. Cl. CPC A61K 36/9066; A61P 25/22; A61P 29/00; A61P 1/00; A61K 31/19; A61K 9/14; A61K 9/48; A61K 36/752; A61K 36/67; A61P 37/00

(57) ABSTRACT

What is described is a formulation comprising curcuma longa, black pepper containing piperine, ginger, magnesium citrate, and lemon juice extract in an amount for reducing the duration and severity of discomfort caused by gluten ingestion. The formulation reduces gastrointestinal inflammation and gastrointestinal upset, improves gut motility, and/or reduces anxiety and irritability.

### CROSS REFERENCE TO RELATED APPLICATION

This application claims the benefit of U.S. Provisional Application No. 62/280,649 filed on Jan. 19, 2016, the entire contents of which are incorporated herein by reference.

### BACKGROUND

For people living with gluten-related disorders, including celiac disease, gluten intolerance, non-celiac gluten sensitivity, and gluten ataxia, a gluten reaction is a digestive, neurological, and/or skin-based response following gluten exposure in the diet. Exposure to gluten, a protein commonly found in wheat, barley, and other grains, may trigger a wide range of symptoms. Symptoms can vary widely among sufferers, but there are a handful of symptoms commonly experienced in gluten-related disorders when the sufferer is exposed to gluten. These include abdominal pain, bloating, nausea, diarrhea or constipation, rash, irritability, anxiety, depression, hyperactivity, fatigue, and insomnia. Reactions commonly last up to five days, with symptoms presenting most intensely within the first 48 hours.

Commonly, treatment for gluten-related disorders is a strict gluten-free diet undertaken to prevent exposure to gluten. When sufferers are exposed to gluten, there are few treatments for their reaction beyond rest, fluids, and a bland diet until symptoms subside. There are over-the-counter medications that offer to provide some relief for individual symptoms or claim to aid gluten digestion. Still, there is an unmet need for a formulation to treat a spectrum of gluten-related disorders and/or to ease the discomforts experienced during a gluten reaction.

### SUMMARY

What is described is a formulation comprising curcuma longa, piperine, ginger, lemon, and magnesium citrate that reduces adverse effects associated with gluten consumption based on reducing gastrointestinal inflammation and gastrointestinal upset, improving gut motility, and reducing anxiety and irritability. What is also described is a method of using the formulation to reduce adverse effects caused by gluten consumption.

### BRIEF DESCRIPTION OF DRAWINGS

The following detailed description may be better understood when viewed in conjunction with the accompanying drawings. Various examples of aspects of the disclosure are shown in the drawings; however, the invention is not limited to the specific methods and instrumentalities disclosed.

FIG. 1 depicts testing results for an embodiment of the dietary supplement disclosed herein.

FIG. 2 depicts results of testing an embodiment of the dietary supplement disclosed herein.

FIG. 3 depicts an alternative view of test results for an embodiment of the dietary supplement disclosed herein.

FIG. 4 depicts symptoms experienced by participants in testing of an embodiment of the dietary supplement disclosed herein.

FIG. 5 depicts an alternative view of test results for an embodiment of the dietary supplement disclosed herein.

### DETAILED DESCRIPTION

Presented herein is a dietary supplement formulation comprising curcuma longa, piperine, ginger, lemon, and magnesium citrate and a method of using the formulation to reduce adverse effects caused by gluten consumption. What is also described is a method of reducing gastrointestinal inflammation, gastrointestinal upset, improving gut motility, and/or reducing anxiety and irritability by administering the formulation. Ingredients in this formulation work together to reduce adverse effects caused by gluten consumption based on reducing gastrointestinal inflammation, gastrointestinal upset, improving gut motility, and reducing anxiety. In contrast to typical treatments for gluten intolerance, the combination of disclosed embodiments is administered subsequent to a gluten reaction to reduce severity and duration of symptoms.

A gluten reaction is believed to involve a response of the body to exposure to a substance containing gluten. Substances containing gluten include grains such as wheat, rye, and barley. It may also be the case that a gluten reaction is triggered by a response of the body to a substance containing a central agonist other than gluten. For example, recent research into gluten sensitivity link gluten reactions to fermentable, poorly absorbed, short-chain carbohydrates (“FODMAPs”). Accordingly, as used herein a gluten reaction includes reactions to gluten, to FODMAPs, or to other as-of-yet unidentified agonists in the constellation of symptoms associated with gluten ingestion by susceptible individuals.

A possible approach to treating or preventing gluten reaction can include prescription or over-the-counter antacids. For example, cimetidine, ranitidine, or other H2 blockers may be prescribed. These can be combined with prescription or over-the-counter pain killers. However, these approaches may have various drawbacks including being ineffectual, expensive, be slow to provide relief, and difficult to procure. They may also provide relief only for specific symptoms, such as gastric pain.

Other possible approaches to treating or preventing gluten reaction include topical oils, probiotics, vitamin supplements, and so forth. However, these too are sometimes ineffectual, expensive, slow to provide relief, or do not provide relief unless taken proactively. They may also be difficult to procure. In addition, many of these approaches may be difficult to carry (e.g. due to weight or size), require refrigeration, or not be shelf-stable.

Another possible approach to treating or preventing gluten reaction involves enzymes. Treatments sometimes comprise ingesting the enzyme with (rather than after) a meal containing gluten. These too are sometimes ineffectual, expensive, or do not provide relief unless taken with or just prior to gluten consumption.

One reason these and other approaches to treatment or prevention of gluten reaction are sometimes ineffectual is that gluten reaction is commonly associated with a wide range of symptoms, including digestive system pain, gastrointestinal bloating, nausea, diarrhea, constipation, fatigue, trouble concentrating, anxiety, depression, brain fog, headaches, sleeplessness, irritability, emotional volatility, rashes, and generalized achiness. Moreover, symptoms associated with gluten reaction typically vary between sufferers. For example, one individual might experience digestive system pain, anxiety, depression, and sleeplessness, while another experiences gastrointestinal bloating, irritability, brain fog, and generalized achiness. Thus, conventional post-exposure treatment is typically selected based on selectively targeting isolated symptoms.

Inflammation is believed by the inventors to be a significant factor in the spectrum of symptoms associated with gluten reaction. The inventors believe there to be a causal relationship between systemic inflammation triggered by gluten or FODMAP exposure and the spectrum of symptoms associated with gluten reaction. Embodiments of the present disclosure alleviate a broad spectrum of symptoms associated with gluten reaction. In an embodiment, a combination of magnesium, curcumin, and ginger, taken together, is believed to result in a reduction of inflammation and prevention of an inflammatory cycle that could result in a range of the symptoms associated with gluten reaction. Embodiments are believed to be more efficacious than the individual ingredients would be, if taken alone. Embodiments further comprise an acidic component, e.g. citric acid, to counter acid-reducing effects of the combination of magnesium, curcumin, and ginger. In some instances, an acidic stomach environment promotes breakdown of long-chain proteins and/or peptides, such as those found in gluten. Without this addition, the acid-reducing effects of the other ingredients in the combination may have negative gastro-intestinal impact on some individuals. It is believed that gluten reaction may be due, in part, to inability or difficulty in digesting long-chain proteins, and that breaking down gluten into shorter chains of proteins and peptides may make gluten more digestible. In addition, those with gluten intolerance or celiac disease are sometimes predisposed to have low levels of stomach acid, and might therefore be more susceptible to negative gastro-intestinal impact without the addition of an acid to the combination of magnesium, curcumin, and ginger.

Embodiments presented herein include a dietary supplement formulation comprising curcuma longa, piperine, ginger, lemon, and magnesium citrate and a method of using the formulation to reduce adverse effects caused by gluten consumption.

Embodiments of the present disclosure are beneficial when taken in response to dietary reactions in addition to those associated with gluten, such as those triggered by allergy or intolerance to other substances, such as those contained in some dairy products. Embodiments of the present disclosure are also useful taken in response to digestive tract inflammation. In each of these cases, the benefits include a reduction in a spectrum of symptoms associated with gluten ingestion by susceptible individuals.

An embodiment of the present disclosure further has the characteristics of being lightweight and transportable, shelf-stable, and comprised of relatively inexpensive substances. These embodiments may, accordingly, be advantageous over other approaches to treatment.

An embodiment of the present disclosure includes supplemental ingredients such as sweeteners, vitamins, probiotics, gelatin, gelatin complex, and flavorings. Testing of sweeteners has included artificial sweeteners, sugar, syrups, and honey. Members of the gluten-sensitive population sometimes have other health issues with sugar. Consequently, sugar is omitted in some embodiments. In other embodiments, a sweetener is provided separately.

Embodiments of the present disclosure are generally related to dietary supplements, and, more particularly, to a formulation that combines ingredients that reduces the severity of adverse reactions from gluten ingestion. For those sensitive to gluten ingestion, anxiety, gastrointestinal inflammation, and gastrointestinal upset (including cramping, bloating, gas, diarrhea, constipation and stomach pain) are all serious conditions in which gluten ingestion appears to play a role. The formulation reduces the severity and duration of gluten reaction symptoms to an extent that is greater than what would be expected based on the known effects of the individual ingredients.

Embodiments of the present disclosure include a dietary supplement composition that can reduce the adverse effects caused by gluten consumption and provide other health benefits. Although it is known that some of the various ingredients have modest benefits for conditions related to the digestive tract, the unique combination of ingredients allows benefits in relation to gluten reaction that are not realized when the ingredients are taken individually. These effects include broader systemic improvement and achievement of results at lower doses than possible with the individual ingredients of the composition.

Curcuma longa contains curcuminoids and turmerones, which are anti-inflammatory agents capable of moderating inflammation associated with gluten ingestion. The extract components are delivered with ground black pepper that improves curcumin bioavailability. The reduction in gut inflammation from the curcuminoids and turmerones decreases the severity and duration of effects caused by gluten insensitivity. Embodiments of the present disclosure comprise curcuma longa. Some embodiments include another compound, or compounds, containing effective amounts of curcuminoids and turmerones. For example, refined curcuminoids and turmerones are added in proportions approximating the proportions of these ingredients in curcuma longa.

Ginger is used in various embodiments to soothe gastrointestinal upset and further to reduce gastrointestinal discomfort. Ginger is also used in some embodiments as a taste masking agent. A taste masking agent, such as ginger, is effective at reducing the taste of the certain ingredients while also soothing gastrointestinal symptoms. In some cases, for example, ginger reduces the sharpness of lemon. Where the taste of an ingredient is not masked, some embodiments include ingredients with unpleasant taste, such as curcuma longa), in a capsule.

Low magnesium levels are sometimes found in those suffering from gastrointestinal inflammation. Magnesium acts as a vasodilator and an anxiety-reducing agent. Magnesium reduces gut spasms and moderates gut motility.

Lemon is itself acidic, but also stimulates the secretion of stomach acid. Low stomach acid levels are associated with gluten insensitivity. Lemon or other similar acidifying agent is included to lessen the acid-reducing effects of magnesium citrate, ginger powder, and curcuma longa. The lemon also acts as a taste masking agent or a flavoring agent.

Possible substitutions for lemon include citric acid, lime, orange, cranberry, and other acidic fruits, fruit juices, and their chemical equivalents.

An embodiment includes curcuma longa. Curcuma longa is provided as curcuma longa extract. Curcuma longa is a rhizomatous herbaceous perennial plant of the Zingiberaceae (ginger) family. The extract is derived from the root of the plant, first by drying and powdering to create turmeric, then further refining by solvent extraction, creating a powder with much higher concentrations of beneficial curcuminoid compounds.

Curcuma longa constituents include three curcuminoids: curcumin (diferuloylmethane, the primary constituent and the one responsible for its vibrant yellow color), demethoxycurcumin and bisdemethoxycurcumin, as well as volatile oils (turmerone, atlantone, and zingiberone), sugars, proteins, and resins. Research has shown curcumin to be a highly pleiotropic molecule capable of interacting with numerous molecular targets involved in inflammation. The various aspects of curcumin include anti-oxidant, anti-hypoglycemic, and anti-inflammatory properties.

Curcuma longa, in various embodiments, acts to reduce general inflammation and provide other health benefits. Curcuma longa containing the anti-inflammatory agents, curcuminoids and turmerones, acts to reduce transitory inflammation in those sensitive to gluten.

An embodiment includes piperine as black pepper that comprises piperine, or piperine in an extracted or purified form. Piperine is a major component of black pepper. Along with its isomer chavicine, piperine is the alkaloid responsible for the pungency of black pepper. Piperine is a natural compound used in the supplement to increase the bioavailability of curcumin. Ground pepper or piperidine significantly improves curcuminoid bioavailability when ingested with curcuma extract allowing for more beneficial systemic effects. Piperine in an extracted or purified form is commercially available. Piperine is extractable from black pepper by a number of chemical means.

An embodiment includes ginger in powdered or liquid form. Ginger powder is created by drying and grinding the entire root of the rhizome of the plant Zingiber officinale. Alternately, juice can be extracted from the root and preserved for use in liquid form. Ginger is known to be used to help digestion and treat stomach upset, diarrhea, and nausea. Embodiments include one or more various forms of ginger, such as powdered ginger, liquid ginger, juiced ginger, ginger extract, and crystalized ginger.

An embodiment includes lemon as juice or crystallized lemon juice. Lemon juice is derived from squeezed whole lemons, and includes oils derived from the rind of the fruit. Crystallized lemon is a powdered form lemon juice that retains properties of fresh lemon juice when rehydrated. Low stomach acid is prevalent in people with gluten intolerance. Lemon juice supports hydrochloric acid in stomach for better digestion. Additionally, lemon juice fights inflammation and affects the rate of absorption in the body.

An embodiment includes magnesium citrate. Magnesium citrate includes a pharmaceutical preparation of magnesium in salt form with citric acid in a 1:1 ratio (one magnesium atom per citrate molecule). Magnesium is involved in more than 300 biochemical reactions in the body, including regulating normal nerve and muscle function. Magnesium acts on many levels in the hormonal axis and regulation of the stress response. In the synapses of the brain, magnesium functions to prevent relay of excitatory impulses. Magnesium citrate acts as an anxiety-relieving agent.

Magnesium citrate also induces an osmotic process by attracting water through the tissues of the gastrointestinal system. Through the osmotic process, magnesium citrate acts in various embodiments as an anti-cramping agent. The magnesium citrate also helps relax bowel spasms due to its effect as a muscle relaxer, to increase stool volume, and to soften stools. In various embodiments, it is made more rapidly effective by delivery in a solution comprising warm water or other liquids at similar temperatures. In some instances, embodiments are delivered at potable warm temperatures. In other instances, embodiments are delivered at cool or cold temperatures.

Magnesium citrate also acts in various embodiments as an effervescent agent. Effervescent magnesium citrate is provided as a water-soluble powder. The effervescent properties of magnesium citrate, in some instances, act as a taste-masking agent.

In an embodiment, magnesium citrate also includes or is provided as a salt such as trimagnesium citrate or trimagnesium dicitrate.

In an embodiment, single dose composition includes an effective amount of curcuma longa extract of 1000 mg to 2000 mg, an effective range of piperine of greater than 20 mg per kg of curcuma longa extract, an effective range of powdered ginger extract from 25 mg to 375 mg, an effective range of magnesium citrate from 2000 mg to 4000 mg, and an effective range of crystalized lemon extract from 3200 mg to 6400 mg.

In an embodiment, a single dose composition includes an effective amount of curcuma longa extract of 1000 mg to 2000 mg, an effective range of piperine of greater than 20 mg per kg of curcuma longa extract, an effective range of powdered ginger extract from 25 mg to 375 mg, an effective range of magnesium from 250 mg to 1000 mg (delivered by magnesium citrate), and an effective range of crystalized lemon extract from 3200 mg to 6400 mg.

In an embodiment, a single dose composition includes an effective amount of curcuma longa extract of 1000 mg to 2000 mg, an effective range of piperine of greater than 20 mg per kg of curcuma longa extract, an effective range of powdered ginger extract from 25 mg to 375 mg, an effective range of magnesium citrate up to 4 g, and an effective range of crystalized lemon extract from 3200 mg to 6400 mg.

In an embodiment, the curcuma longa, piperine, ginger, lemon, and magnesium citrate composition is packed as single dose units. The active ingredients rapidly dissolve or disperse in the gastrointestinal tract to achieve uptake and moderate the environment in the gastrointestinal tract. A single dose composition as described herein is effective for individuals weighing at least 100 pounds. A single dose composition for individuals weighing less than 100 pounds is obtained by adjusting dosage according to the weight of the individual.

In an embodiment, a single dose composition includes at least 1 gram curcuma longa, at least 0.02 grams piperine, at least 0.025 grams powdered ginger extract, at least 2 grams magnesium citrate, and at least 3.2 grams of crystalized lemon extract. The single dose composition may be adjusted proportionally by weight for individuals weighing less than 100 pounds.

In another embodiment, a nutritional supplement comprises 1.0 part by weight curcuma longa, at least 0.02 parts by weight piperine, at least 0.025 parts by weight powdered ginger extract, at least 2 parts by weight magnesium citrate, and at least 3.2 parts by weight of crystalized lemon extract.

In another embodiment, powdered ginger extract is substituted with alternative ginger forms such as those described herein, adjusting for weight and concentration of the substituted form.

In another embodiment, curcuma longa is substituted with alternative curcuma longa forms such as those described herein, adjusting for weight and concentration of the substituted form.

In another embodiment, crystalized lemon extract is substituted with alternative lemon forms such as those described herein, adjusting for weight and concentration of the substituted form.

In another embodiment, magnesium citrate is substituted with alternative magnesium citrate forms such as those described herein, adjusting for weight and concentration of the substituted form.

In another embodiment, piperine is substituted with alternative piperine forms such as those described herein, adjusting for weight and effectiveness of the substituted form.

In another embodiment, a dose is delivered with additional flavorings, vitamins, and/or liquids.

In another embodiment, a dose consists of two formulations. The first comprises an effective range of 1000 mg to 2000 mg curcuma longa extract and an effective range of piperine of greater than 20 mg per kg concentration, optionally in a capsule. The second comprises an effective range of powdered ginger extract from 25 mg to 375 mg, an effective range of magnesium citrate from 2 g to 4 g, and an effective range of crystalized lemon extract from 3.2 g to 6.4 g, optionally in a powder to be mixed with water.

In another embodiment, a nutritional supplement comprises two formulations. The first comprises 1.0 part by weight curcuma longa to at least 0.02 parts by weight piperine. The second comprises at least 0.025 parts by weight powdered ginger extract, at least 2 parts magnesium citrate by weight, and at least 3.2 parts crystalized lemon extract by weight.

An embodiment comprises additional taste masking agents or flavors. The flavors of components with strong or objectionable flavors, such as curcuma longa, are masked by including them in a capsule. Components with desired or non-objectionable flavor are provided as a liquid or powder to be mixed with liquid.

In another embodiment, the formulation thereof is a tablet of compressed powder that is mixed with water or an aqueous liquid for consumption.

In an embodiment, the supplement is provided as a single capsule. Embodiments incorporate measures to prevent the recipient from perceiving objectionable flavors. For example, the capsule is encased in a gelatin or other coating.

In an embodiment, effervescence is increased to provide greater taste masking. In some embodiments, effervescence provides a more rapid soothing effect or increases the soothing effect. Effervescence may be provided by magnesium citrate, sodium bicarbonate, and so forth. In liquid delivery forms, carbonation may be used to provide effervescence.

In an embodiment, gelatin, vegetable collagen, or other collagen is included as a powder, or by providing the product as a gelatin cube. Gelatin can be provided in an embodiment to improve effectiveness by providing a feeling of bulk or fullness to the stomach, which is effective for reducing gastrointestinal symptoms.

In an embodiment, chamomile or other sleep-inducing agents are included. Some embodiments include added vitamins such as vitamins C and B. Embodiments have been tested with a vitamin complex that included each of the following: vitamin C (as ascorbic acid, zinc ascorbate) 1000 mg; thiamin (as thiamin hydrochloride)(VitB1) 0.38 mg; riboflavin (as riboflavin-5-phosphate) (VitB2) 0.43 mg; niacin (VitB3) 4 mg; vitamin B6 (as pyridoxine hydrochloride) 10 mg; folic acid (VitB9) 100 mcg; vitamin B12 (as cyanocobalamin) 25 mcg; pantothenic acid (as calcium pantothenate) (VitB5); calcium (as calcium carbonate, monobasic calcium phosphate, tribasic calcium phosphate, calcium pantothenate) 50 mg; phosphorus (as monobasic potassium phosphate, monobasic calcium phosphate, monobasic sodium phosphate, tribasic calcium phosphate) 38 mg; zinc (as zinc ascorbate) 2 mg; manganese (as manganese gluconate) 60 mg; chromium (as chromium picolinate) 10 mcg; sodium (as sodium bicarbonate, monobasic sodium phosphate) 65 mg; and potassium (as potassium bicarbonate, potassium carbonate, monobasic potassium phosphate) 200 mg. Embodiments comprising vitamins are believed to remain efficacious when used in these or lesser amounts. Some embodiments also include various probiotics, gelatins, and gelatin complexes and are also believed to remain efficacious.

Embodiments of the present disclosure can be used in conjunction with the treatment of various conditions that involve a spectrum of symptoms similar to those of gluten reaction, including gastrointestinal inflammation and gastrointestinal upset, impaired gut motility, anxiety and irritability. Conditions such as, or similar to, what has been described as “leaky gut syndrome” have been suggested to be similar in origin to gluten-related disorders. Embodiments offer similar relief of symptoms to patients with leaky gut syndrome or other conditions presenting with similar symptoms.

An embodiment comprises a formulation that reduces adverse effects caused by gluten consumption based on reducing gastrointestinal inflammation and gastrointestinal upset, improving gut motility, and/or reducing anxiety and irritability, comprising curcuma longa, black pepper, ginger, lemon, and magnesium citrate.

In an embodiment, the curcuma longa of the formulation comprises curcumin, demethoxycurcumin, and bisdemethoxycurcumin.

In an embodiment, the curcuma longa of the formulation comprises curcuma longa extract. In an embodiment, the curcuma longa of the formulation comprises an anti-inflammatory agent or otherwise provide an anti-inflammatory effect. In an embodiment, the curcuma longa of the formulation comprises curcuminoids and turmerones.

The anti-inflammatory effect provided by the curcuma longa can be substituted with another substance containing anti-inflammatory properties.

In an embodiment, the piperine of the formulation is supplied as black pepper (piper nigrum). In an embodiment, long pepper (piper longum) fruit is used.

In an embodiment, the ginger of the formulation masks the taste of the curcuma longa. Accordingly, in various embodiments the concentration of ginger in an embodiment is selected based on its taste-masking properties.

In an embodiment, the lemon of the formulation comprises at least one of lemon juice, crystallized lemon juice, or citric acid. The lemon of the formulation is used in some embodiments to mask the taste of curcuma longa.

In an embodiment, the magnesium citrate is provided as an effervescing agent. The effervescence resulting from the effervescing agent is used for taste-masking, and in some embodiments is used to permit reduced amounts of other taste-masking ingredients, such as ginger.

In an embodiment, the formulation comprises a dosing unit of 1000 mg to 2000 mg curcuma longa extract, piperine in concentration of at least 20 mg per kilogram, 25 mg to 375 mg powdered ginger extract, 2 g to 4 g magnesium citrate, and 3200 mg to 6400 mg crystallized lemon extract.

Embodiments of the formulation include various combinations of the instances described above. The described instances should thus not be construed as being mutually exclusive, and the inclusion of any one of the above-described instances will not preclude the inclusion of any other of the above-described instances.

In an embodiment, a formulation for treating a subject comprises a first part comprising 1000 mg to 2000 mg curcuma longa extract and at least 20 mg piperine and a second part separate from the first part, the second part comprising 25 mg to 375 mg powdered ginger extract, 2 g to 4 g magnesium citrate, and 3200 mg to 6400 g crystallized lemon extract.

In some instances, the first part of the above formulation is provided in a capsule form.

In some instances, the second part of the above formulation is provided as a powder. In some instances, the second part of the above formulation is mixed in liquid prior to ingestion. In some embodiments, for example, the second part is provided in a bag or pouch, while the first part is provided as a capsule.

Various embodiments of the formulation include combinations of the instances described above. The described instances should thus not be construed as being mutually exclusive, and the inclusion of any one of the above-described instances will not preclude the inclusion of any other of the above-described instances.

A method for treating gluten reaction comprises orally administering a dose comprising 1000 mg to 2000 mg curcuma longa extract, piperine in concentration of at least 20 mg per kilogram, 25 mg to 375 mg powdered ginger extract, 2 g to 4 g magnesium citrate, and 3.2 g to 6.4 g crystallized lemon extract.

An embodiment of the method comprises orally administering the dose subsequent to a gluten reaction, or subsequent to consumption of food associated with gluten reaction. Embodiments of the method therefore beneficially involve ingestion of the formulation after the exposure to gluten has already occurred or after the onset of symptoms, in contrast to other approaches to gluten intolerance and gluten reaction.

An embodiment of the method comprises orally administering the dose as a single unit.

An embodiment of the method comprises orally administering the dose in separate units, as described herein.

Embodiments of the method include various combinations of the instances described above. The described instances should thus not be construed as being mutually exclusive, and the inclusion of any one of the above-described instances will not preclude the inclusion of any other of the above-described instances.

An embodiment of a dietary supplement comprises 1.0 part by weight curcuma longa, at least 0.02 parts by weight piperine, at least 0.025 parts by weight powdered ginger extract, at least 2 parts by weight magnesium citrate, and at least 3.2 parts by weight of crystalized lemon extract.

An embodiment formulation that reduces gastrointestinal inflammation and gastrointestinal upset comprises a curcuma longa portion, piperine portion, ginger portion, lemon portion, and a magnesium citrate portion. The formulation comprises the equivalent of 1.0 part by weight curcuma longa, at least 0.02 parts by weight piperine, at least 0.025 parts by weight powdered ginger extract, at least 2 parts by weight magnesium citrate, and at least 3.2 parts by weight of crystalized lemon extract.

In an embodiment, the curcuma longa portion is provided by an equivalent effective amount of a substance comprising curcuma longa, such as cumin. The ginger portion is provided by an equivalent effective amount of a ginger product. The lemon portion is provided by various forms of lemon, other citrus products, or other acidic fruits such as pomegranate, pineapple, kiwi, and cranberry. The magnesium citrate portion is provided by an equivalent effective amount of magnesium in salt form with citric acid in a 1:1 ratio, or as an equivalent effective amount of trimagnesium citrate. In some embodiments, magnesium hydroxide or magnesium sulfate may be used. The total amount of the lemon portion may be adjusted depending on the amount of citric acid provided via the magnesium portion, in order to reduce any negative impact on digestion due to an antacid effect of the magnesium citrate portion.

The terms “comprising,” “including,” “having,” and the like are synonymous and are used inclusively, in an open-ended fashion, and do not exclude additional features and/or steps not explicitly stated. The term “or” is used in its inclusive sense. When used in a list of features and/or steps, “or” means one or more elements of the list, rather than all elements of the list.

Certain embodiments are described herein. The described embodiments are presented by way of example, and should not be construed as limiting the scope of the present disclosure to only those embodiments described. Unless explicitly indicated, nothing in the present disclosure is intended to imply that a given feature and/or step is necessary or indispensable.

### EXAMPLES

### Example 1

The following formulations were made (Table 1)

| ingredient | Part by weight | weight | |
|---|---|---|---|
| curcuma longa | 1.0 | 1000 to 2000 | mg |
| piperine | 0.02 | 20 | mg/kg |
| ginger | 0.025 | 25 to 375 | mg |
| magnesium citrate | 2 | 2 to 4 g (delivering at | |
| | | least 250 mg magnesium) | |
| lemon extract | 3.2 | 3200 to 6400 | mg |

### Example 2

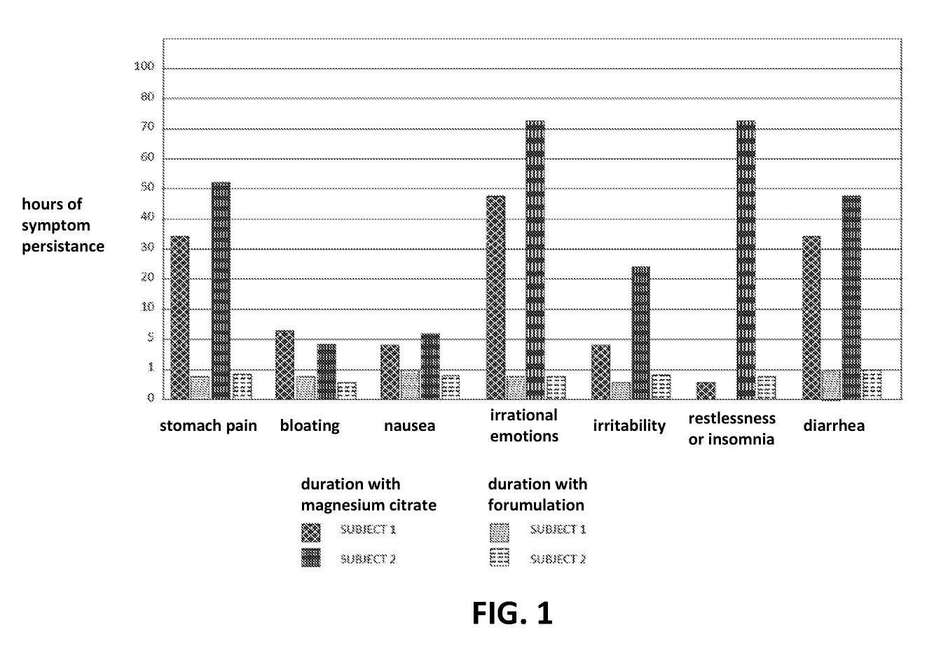

FIG. 1 depicts testing results for an embodiment of the dietary supplement disclosed herein. Formulations as described in Example 1 were used. As can be seen from FIG. 1, test results indicate a reported reduction in the typical duration of a variety of symptoms associated with gluten reaction.

The test rules depicted by FIG. 1 were obtained by survey of two test subjects. Testing proceeded in two phases. In the first phase, subsequent to the onset of symptoms associated with gluten reaction, the test subjects were surveyed to collect information pertinent to the duration of the associated symptoms. The first phase was conducted without ingestion of the formulation, but with a magnesium citrate dosage. In a second phase, an embodiment of the dietary supplement disclosed herein was administered. The supplement comprised curcuma longa, piperine, ginger, lemon, and magnesium citrate. The supplement was taken by the test subjects subsequent to the onset of symptoms associated with ingesting gluten. The test subjects were again surveyed to collect information pertinent to the duration of the associated symptoms. As depicted in FIG. 1, both test subjects experienced reduced duration of associated symptoms when the supplement was taken subsequent to the onset of the symptoms, compared to magnesium citrate.

Information was also collected from a third test subject (results not depicted in FIG. 1). In the first phase, the test subject consumed lemon diluted in water following the onset of symptoms associated with gluten reaction. In the second phase, the test subject consumed the formulation following the onset of symptoms associated with gluten reaction. The test subject reported significantly reduced severity and duration of symptoms during the second phase, as compared to the first phase.

### Example 3

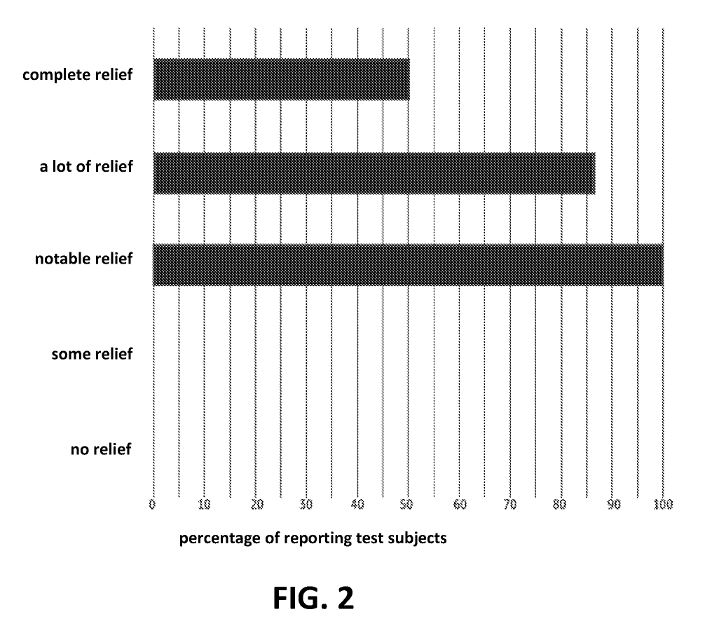

FIG. 2 depicts results of testing an embodiment of the dietary supplement disclosed herein. The testing comprised data collection from individuals who self-identified as being intolerant to gluten or having been diagnosed with celiac disease. Participants included both males and females of various ages.

Participants were provided with samples of the disclosed dietary supplement. Participants were instructed to ingest the provided sample as soon as possible after ingesting gluten or experiencing the onset of symptoms associated with gluten reaction. Participants were further requested to complete a survey comprising questions related to the nature of their experience with gluten reaction, their symptoms, whether ingestion of the sample provided relief, and if so the nature and speed of the relief provided.

Of the participants, there were twenty-two responses to the survey. Asked to rate the amount of relief provided on a 1-5 sale, with ‘1’ corresponding to “no relief” and ‘5’ corresponding to “complete relief,” eleven reported ‘5’ (“complete relief”), eight reported ‘4’ (“a lot of relief”), and three reported ‘3’ (“notable relief”). None of the twenty-two survey responses reported ‘2’ (“little relief”) or 1 (“no relief”).

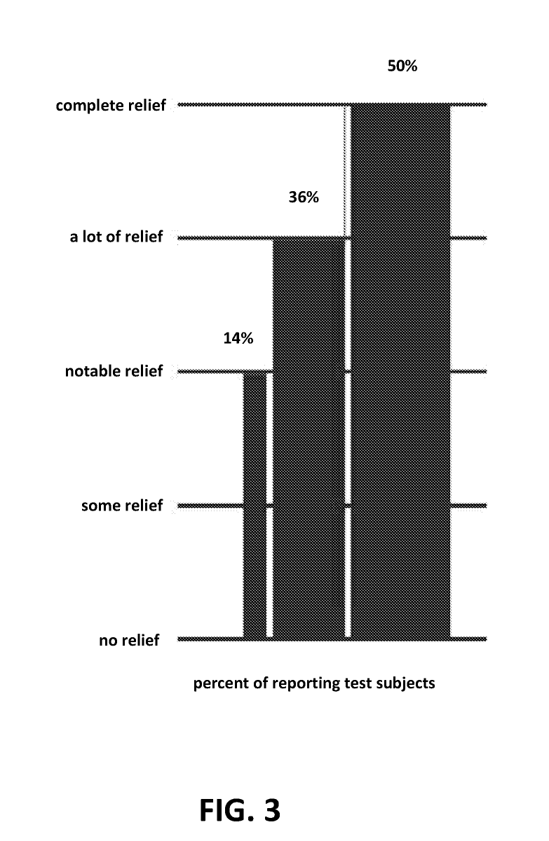

FIG. 2 depicts results of this survey. It shows that 50% of the test subjects who completed the survey indicated that the sample provided at least “complete relief” to one or more of their symptoms. Likewise, FIG. 2 shows that 86% of the responses reported at least “a lot of” relief, and 100% of the responses reported at least “notable relief” FIG. 3 depicts an alternative view of the test results just described.

### Example 4

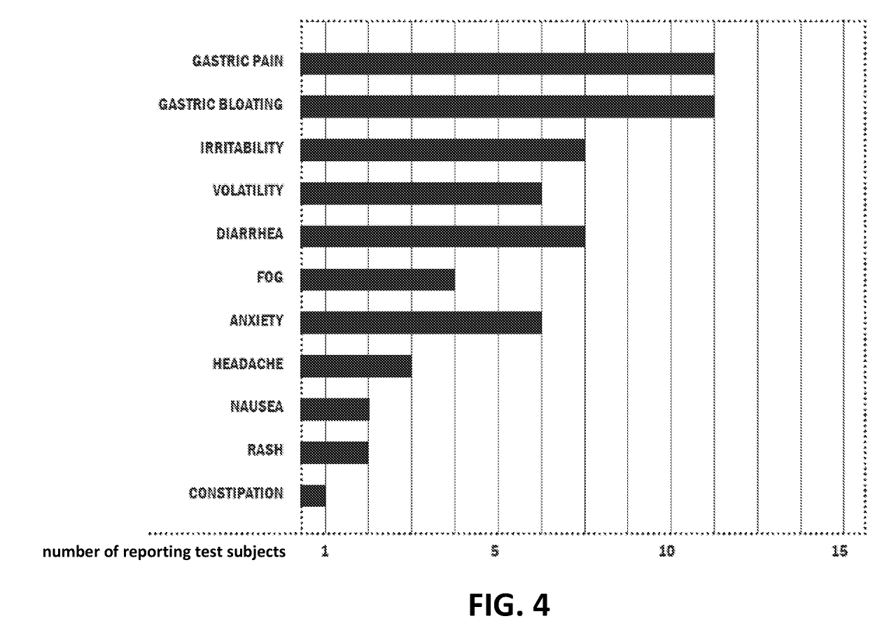

FIG. 4 depicts symptoms experienced by participants in the testing of an embodiment of the dietary supplement disclosed herein. As noted in the figure, the test subjects reported a wide range of symptoms including gastric pain, gastric bloating, irritability, volatility, diarrhea, brain fog, anxiety, headache, nausea, rash, and constipation. Of these, gastric pain and gastric bloating were most common.

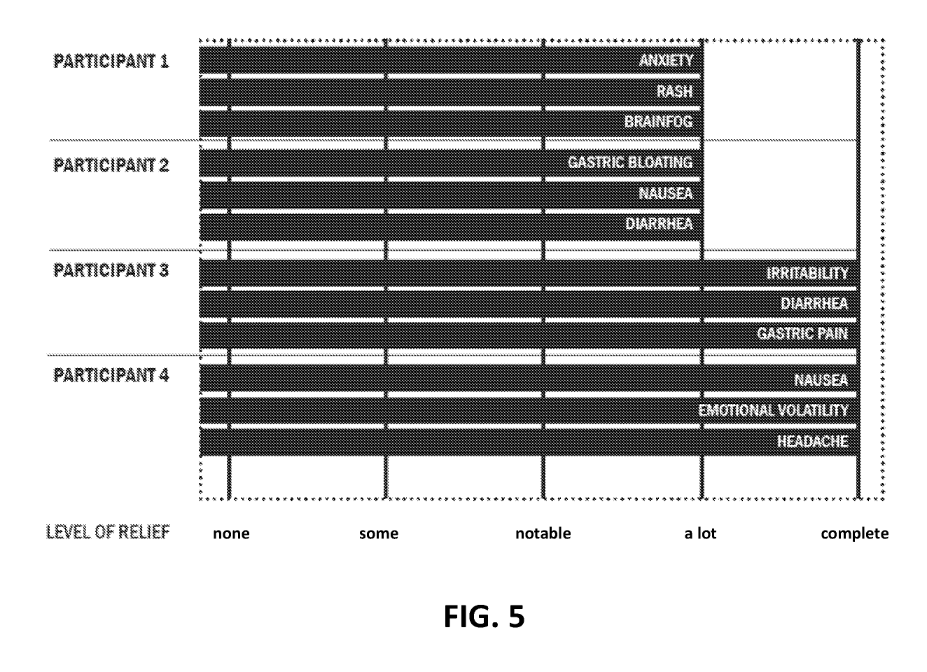

FIG. 5 depicts an alternative view of the test results. In particular, FIG. 5 depicts symptoms experienced by each of four selected test participants. For example, participant 1 experienced a cluster of symptoms including anxiety, rash, and brain fog, while participant 2 experienced a cluster of symptoms including gastric bloating, nausea, and diarrhea. FIG. 5 also depicts the level of relief experienced by each of the selected participants. As depicted, the level of relief experienced by each participant tended to be similar across symptoms. For example, participant 2 reported “quite a lot” of relief from each of the reported symptoms of gastric bloating, nausea, and diarrhea, and participant 3 reported “complete relief” for each of the reported symptoms of irritability, diarrhea, and gastric pain.

Of the test subjects who responded to the survey, ten reported gastric bloating, nine reported gastric pain, seven reported emotional volatility, seven reported irritability, five reported anxiety, and five reported diarrhea.

Of the test subjects who responded to the survey, time to relief was reported as follows: 5 minutes—9%; 10 minutes—5%; 15 minutes—14%; 20 minutes—9%; 30 minutes—36%; 45 minutes—18%; and 60 minutes or more—9%. Thus, 91% of responding test subjects reported relief within 45 minutes of ingesting the sample, and 73% reported relief within 30 minutes of ingesting the sample. No negative reactions were reported.

What is claimed is:

1. A formulation comprising 1.0 part by weight curcuma longa, at least 0.02 parts by weight piperine, at least 0.025 parts by weight powdered ginger extract, at least 2 parts by weight magnesium citrate, and at least 3.2 parts by weight of crystalized lemon extract.

2. The formulation of claim 1, comprising a dosing unit of 1000 mg to 2000 mg curcuma longa extract, piperine in concentration of at least 20 mg per kilogram, 25 mg to 375 mg powdered ginger extract, 2 g to 4 g magnesium citrate, and 3200 mg to 6400 mg crystallized lemon extract.

3. The formulation of claim 2, wherein the dosing unit is provided as a first part comprising the curcuma longa extract and the piperine, and a second part separate from the first part, the second part comprising the powdered ginger extract, the magnesium citrate, and the crystallized lemon extract.

4. The formulation of claim 3, wherein the first part is provided in a capsule and the second part is provided as a powder.

5. The formulation of claim 3, wherein the second part is mixed in liquid prior to ingestion.

6. A formulation that reduces adverse effects caused by gluten consumption, the formulation comprising curcuma longa, black pepper, ginger, lemon, and magnesium citrate.

7. The formulation of claim 6, wherein the curcuma longa comprises curcumin, demethoxycurcumin, and bisdemethoxycurcumin.

8. The formulation of claim 6, wherein the curcuma longa is curcuma longa extract.

9. The formulation of claim 6, wherein the curcuma longa comprises an anti-inflammatory agent.

10. The formulation of claim 6, wherein the curcuma longa comprises curcuminoids and turmerones.

11. The formulation of claim 6, wherein the black pepper comprises piperine.

12. The formulation of claim 6, wherein the magnesium citrate is an effervescing agent.

13. The formulation of claim 6, wherein the lemon consists of at least one of lemon juice, crystallized lemon juice, or citric acid.

14. The formulation of claim 6, comprising a dosing unit of 1000 mg to 2000 mg curcuma longa extract, piperine in concentration of at least 20 mg per kilogram, 25 mg to 375 mg powdered ginger extract, 2 g to 4 g magnesium citrate, and 3200 mg to 6400 mg crystallized lemon extract.

15. A formulation for treating a subject comprising a first part comprising 1000 mg to 2000 mg curcuma longa extract and at least 20 mg piperine and a second part separate from the first part, the second part comprising 25 mg to 375 mg powdered ginger extract, 2 g to 4 g magnesium citrate, and 3200 mg to 6400 g crystallized lemon extract.

16. A method for treating gluten reaction, the method comprising orally administering a dose comprising 1000 mg to 2000 mg curcuma longa extract, piperine in concentration of at least 20 mg per kilogram, 25 mg to 375 mg powdered ginger extract, 2 g to 4 g magnesium citrate, and 3.2 g to 6.4 g crystallized lemon extract.

17. The method of claim 16, further comprising orally administering the dose subsequent to a gluten reaction.

18. The method of claim 16, further comprising administering the dose subsequent to consumption of food associated with gluten reaction.

19. The method of claim 16, wherein the powdered ginger extract is added in proportion to the curcuma longa to mask taste of the curcuma longa.

20. The method of claim 16, wherein the curcuma longa extract, the piperine, the powdered ginger extract, the magnesium citrate, and the crystallized lemon extract is supplied as a single dose unit.

21. A dietary supplement that reduces gastrointestinal inflammation and gastrointestinal upset, the formulation comprising a curcuma longa portion, piperine portion, ginger portion, lemon portion, and magnesium portion.
